(19) United States
(12) Patent Application Publication
(10) Pub. No.: US 2013/0096646 A1
(43) Pub. Date: Apr. 18, 2013

(54) Battery Ventilation for a Medical Device

(71) Applicants: Altan Yildirim, Innsbruck (AT); Bernhard Jamnig, Innsbruck (AT); Alexander Duftner, Innsbruck (AT)

(72) Inventors: Altan Yildirim, Innsbruck (AT); Bernhard Jamnig, Innsbruck (AT); Alexander Duftner, Innsbruck (AT)

(73) Assignee: MED-EL ELEKTROMEDIZINISCHE GERAETE GMBH, Innsbruck (AT)

(21) Appl. No.: 13/611,558

(22) Filed: Sep. 12, 2012

Related U.S. Application Data
(60) Provisional application No. 61/533,491, filed on Sep. 12, 2011.

(51) Int. Cl. A61N 1/378; A61N 1/36

(52) U.S. Cl. CPC A61N 1/3787; A61N 1/36032
USPC 607/57; 607/61

(57) ABSTRACT

A medical device is presented that includes an external portion adapted for placement external to the skin of a user. The external portion includes a battery pack for interfacing with at least one battery cell. The battery pack includes a housing, the housing defining air inlet and/or outlet holes such that fluid flow is enabled through at least a part of the housing. A micro-ventilation mechanism moves air through at least a part of the housing.

### CROSS-REFERENCE TO RELATED APPLICATIONS

This application claims priority from U.S. provisional patent application Ser. No. 61/533,491 filed Sep. 12, 2011, entitled “Battery Ventilation for a Medical Device,” which is incorporated herein by reference in its entirety.

### TECHNICAL FIELD

The present invention relates to battery ventilation systems and methodologies, and more particularly to battery ventilation systems and methodologies for a medical device, such as a hearing implant system.

### BACKGROUND ART

Medical device/implant systems, such as a hearing aid, a laryngeal pacemaker or a hearing implant system (e.g., a cochlear or middle ear implant), often include one or more high performance batteries for the supply of power. Typically, these batteries, for example, a Zn-air battery, require air flow into a battery housing for chemical reaction necessary for operation. In some cases, air flow is also needed to dissipate heat and provide cooling. To enable the air flow, the device/battery housing often includes various holes that allow air to circulate to and from the external environment.

However, the holes on the housing can be disadvantageous and create design challenges. To provide sufficient circulation of air, the position and size of the holes are often a hard requirement that can influence the internal and external design of the device housing. The holes may affect the mechanical stability of the battery housing, particularly if the walls of the housing are thin. Sweat and dirt can come through the holes which may cause corrosion and non-hygienic conditions. FIG. 1 illustratively shows corrosion on battery contacts 101 due to sweat and voltage.

FIG. 2 shows a conventional battery pack 201 associated with a cochlear prosthesis, while FIG. 3 shows a top view of the battery pack. A cochlear prosthesis essentially includes two parts, the speech processor (also referred to as an audio processor) and the implanted stimulator. The speech processor (which may be a Behind the Ear (BTE) device, but also may be, without limitation, a button processor device) typically includes the power supply (battery pack with associated batteries) of the overall system and a processor, which may be a microprocessor, used to perform signal processing of the acoustic signal to extract the stimulation parameters. The implanted stimulator generates the stimulation patterns and conducts them to the nervous tissue by means of an electrode array which usually is positioned in the scala tympani in the inner ear. The connection between speech processor and stimulator is established either by means of a radio frequency link (transcutaneous) or by means of a plug in the skin (percutaneous).

The battery pack 201 attaches to speech processor 203. Air inlet holes 205 are positioned on the housing of battery pack 201. Two additional holes are found on the lower opposite of the battery pack housing. Air goes in through the holes and flows to the batteries through a channel, as indicated by the arrows. The air holes 205 on the housing are placed and designed so that the batteries receive sufficient air via the holes 205. However, any optimization of the holes 205 must not risk stability of the battery pack's mechanical structure. For example, the thickness of the housing is determined, in part, by the size of the air channel(s). Furthermore, it is better to place the holes on the outer side of the battery pack away from the head so that they do not come in direct contact with the hair and skin, and consequently with sweat and other chemicals. Additional miniaturization of the battery pack 201, for example, a slimmer housing, is difficult as it is problematic to find optimal placement of air holes or air channels through the housing while maintaining mechanical stability. For example, the battery pack 201 could have smaller dimensions if the air channel(s) is allowed to be narrower.

### SUMMARY OF THE EMBODIMENTS

In accordance with a first embodiment of the invention, a medical device and methodology includes an external portion adapted for placement external to the skin of a user. The external portion includes a battery pack for interfacing with at least one battery cell. The battery pack includes a housing, the housing defining air inlet and/or outlet holes such that fluid flow is enabled through at least a part of the housing. A micro-ventilation mechanism moves air through at least a part of the housing.

In accordance with related embodiments of the invention, the medical device may further include an implantable portion that receives a signal from the external portion. The external portion may include a first coil, with the implantable portion including a second coil, the first coil and the second coil for transcutaneous transmission of the signal via electromagnetic coupling. For example, the battery pack may supply a power signal to the first coil, for transcutaneous transmission to the second coil. The implantable portion may include a stimulator module for producing for the auditory system of a user a stimulation representative of an acoustic signal. The stimulation may be an electrical stimulation and/or a mechanical stimulation.

In related embodiments of the invention, the external portion may further include a processor module, the micro-ventilation mechanism moving air across the processor module. The micro-ventilation mechanism may be a Microelectromechanical Systems (MEMS) device. The micro-ventilation mechanism may act as a fluid pump and/or fan. The micro-ventilation mechanism may include a membrane.

In further related embodiment of the invention, the battery pack may provide power to the micro-ventilation mechanism. The external portion may include a solar cell for providing power to the micro-ventilation mechanism. The external portion may include a thermoelectric generator module for providing power to the micro-ventilation mechanism.

In still further embodiments of the invention, the micro-ventilation mechanism may be electronically passive. The micro-ventilation system may include a movable mass, which may wind a spring. The movable mass may soak at each movement a volume of air. The movable mass may rotate. The movable mass may be part of the housing.

In yet further embodiments of the invention, the mass of the micro-ventilation mechanism may be below 1 gram, or below 0.5 gram. The medical device may be a hearing aid, a cochlear implant, and/or a laryngeal pacemaker.

In accordance with another embodiment of the invention, a medical device and methodology includes an external portion adapted for placement external to the skin of a user. The external portion includes a housing, a battery pack, and a micro-ventilation mechanism. The battery pack interfaces with at least one battery cell. The housing defines air inlet and/or outlet holes such that fluid flow is enabled through at least a part of the housing. The micro-ventilation mechanism moves air through at least a part of the housing.

In accordance with related embodiment of the invention, the battery pack may include a battery pack housing, with the micro-ventilation mechanism positioned within the battery pack housing. The battery pack housing may be coupled to, or integral with, a housing associated with a speech processor or other electronics. In alternative embodiments, the micro-ventilation mechanism may be positioned within the housing associated with the speech processor or other electronics, but external to the battery pack housing.

### BRIEF DESCRIPTION OF THE DRAWINGS

The foregoing features of embodiments will be more readily understood by reference to the following detailed description, taken with reference to the accompanying drawings, in which:

FIG. 1 (prior art) illustratively shows corrosion on battery contacts of a battery pack due to sweat and voltage;

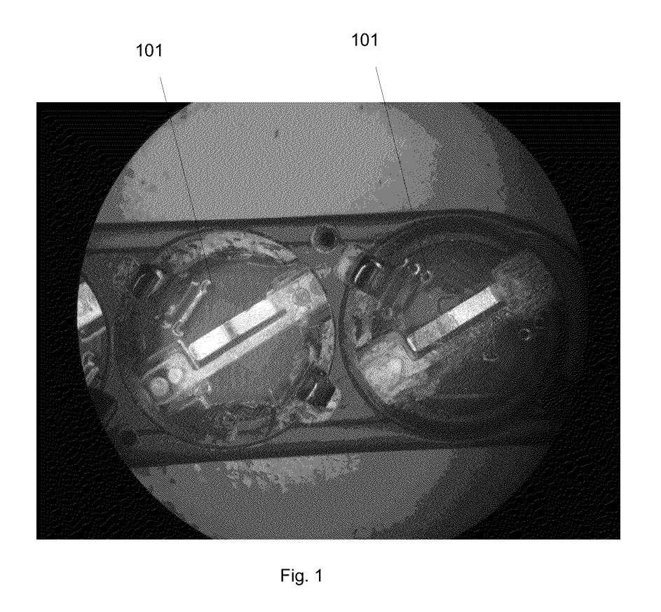

FIG. 2. (prior art) shows a conventional battery pack associated with a cochlear prosthesis;

FIG. 3 (prior art) shows a top view of the battery pack depicted in FIG. 2;

FIGS. 4(a) and (b) (prior art) show a MEMS microturbine and microengine respectively.

FIGS. 5(a-c) show various placements of a battery, micro-ventilation mechanism, and/or processor of a medical device, in accordance with various embodiments of the invention.

FIGS. 6(a-f) show positions of air holes relative to a battery, a micro-ventilation mechanism, and/or a processor, in accordance with various embodiments of the invention.

FIG. 7 shows an electronically passive micro-ventilation mechanism that includes a rotatable mass, in accordance with various embodiments of the invention.

### DETAILED DESCRIPTION OF SPECIFIC EMBODIMENTS

Definitions. As used in this description and the accompanying claims, the following terms shall have the meanings indicated, unless the context otherwise requires:

“Battery Pack” may include any number of battery cells, including a single battery cell. If a plurality of battery cells are utilized, they may be configured, without limitation, in serial, parallel, or a combination of series and parallel.

In illustrative embodiments, a medical device and methodology is presented that includes a micro-ventilation mechanism for moving air across a battery pack and/or various electronics. The medical device may be, for example, a hearing aid, a hearing implant such as a cochlear implant or a middle ear implant, or a laryngeal pacemaker. Details are discussed below.

Use of a micro-ventilation mechanism advantageously allows ventilation holes on the housing associated with the battery pack or other electronics to be optimally sized and placed. For example, by using a micro-ventilation mechanism, the ventilation holes on the housing can be made smaller (compared to when no micro-ventilation mechanism is used). Furthermore, the use of such a micro-ventilation mechanism may allow for a filter or grid to be placed in the holes to prevent entry of, without limitation, dust or sweat. Without a micro-ventilation mechanism, a filter or grid adds complexity since it may reduce the air flow rate, which will affect battery performance. The medical device with the micro-ventilation mechanism may advantageously be used in dusty or hot environments. In such environments, larger holes would collect more dust and sweat compared to smaller holes with or without a filter.

The micro-ventilation mechanism may be used to regulate and move fluid, such as air, across, without limitation, batteries and/or other electronics within a housing associated with the medical device. Electronics may include, for example, a microprocessor, digital signal processing components, filters, and/or memory.

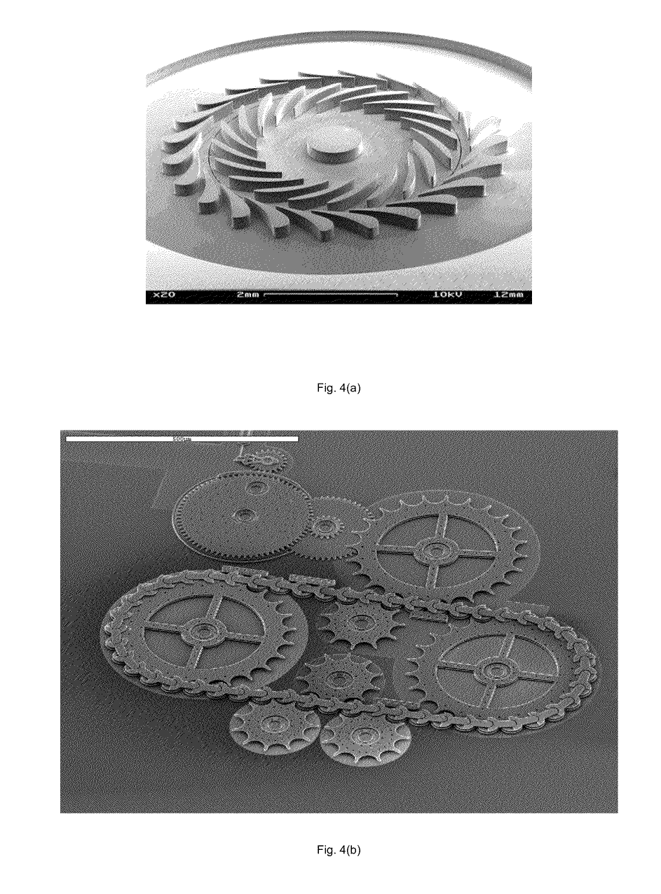

The micro-ventilation mechanism may be, without limitation, an air pump, a fan, a blower, a Microelectro-mechanical Systems (MEMS), and/or fabricated using a membrane technique. FIGS. 4(a) and (b) show a MEMS microturbine and microengine respectively. Since it is a tiny mechanism, (typically MEMS are made up of components between 1 to 100 micrometers in size (i.e. 0.001 to 0.1 mm) and MEMS devices generally range in size from 20 micrometers (20 millionths of a meter) to a millimeter), it may only consume a small amount of energy and pump a small amount of air that is sufficient for the batteries and/or electronics. The sufficient amount of airflow may be determined while the device is operating, and the micro-ventilation mechanism maybe adjusted when in use, for the required power. For example, the micro-ventilation mechanism may also be adjusted such that it switches on or regulates its speed automatically when a higher rate of air-flow is necessary. The described ability to move air to the batteries gives more freedom in the design and the placement of the holes and the path for the air flow. Therefore, the battery pack can be designed to have smaller dimensions.

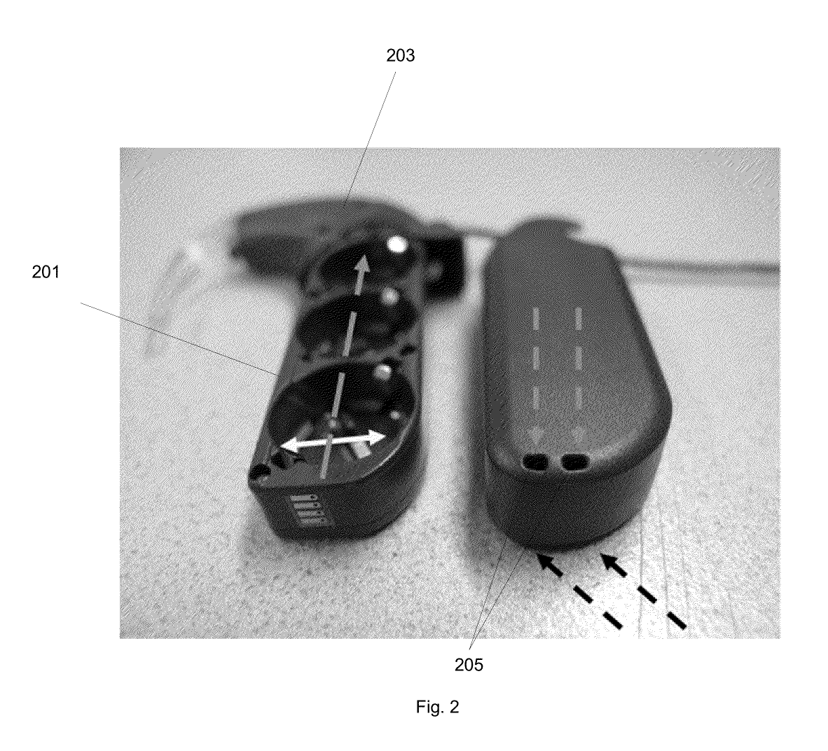

As noted above, air that is moved across the battery pack may also be used for the cooling of a processor or other electronics (such as, for example, the inductive coil of a speech processor, not shown in FIG. 2). Therefore a more compact processor structure can be built.

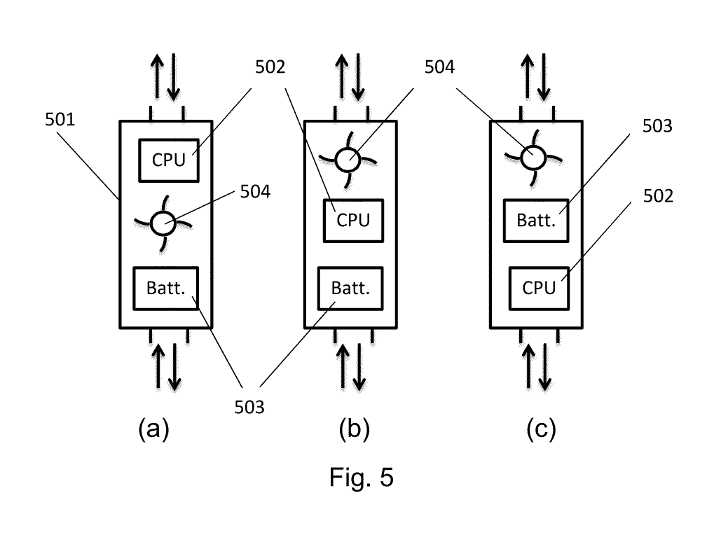

FIGS. 5(a-c) show various placements in relation to the cooling air-stream of a battery pack 503, micro-ventilation mechanism 504, and/or processor 502 of a speech processor 501, in accordance with various embodiments of the invention. Placements of the battery pack 503, micro-ventilation mechanism 504, and/or the processor 502 may be placed in an optimum way, considering the direction of dirt, sweat, heat transport and water flow. It is to be understood that the micro-ventilation mechanism 504 may be positioned in any desired position within the speech processor housing. For example, the micro-ventilation mechanism 504 may be positioned within the battery pack housing (that may be attachable to, integral with, or otherwise positioned within, the speech processor housing). Alternatively, and without limitation, the micro-ventilation mechanism may be positioned outside of the battery pack housing (in various embodiments, the battery pack may not have its own housing) in a desired location within the speech processor housing. More particularly, FIG. 5(a) shows the micro-ventilation mechanism 504 placed between the processor 502 and the battery pack 503; FIG. 5(b) shows the processor 502 placed between the micro-ventilation mechanism 504 and the battery pack 503; and FIG. 5(c) shows the battery pack 503 placed between the processor 502 and the micro-ventilation mechanism 504.

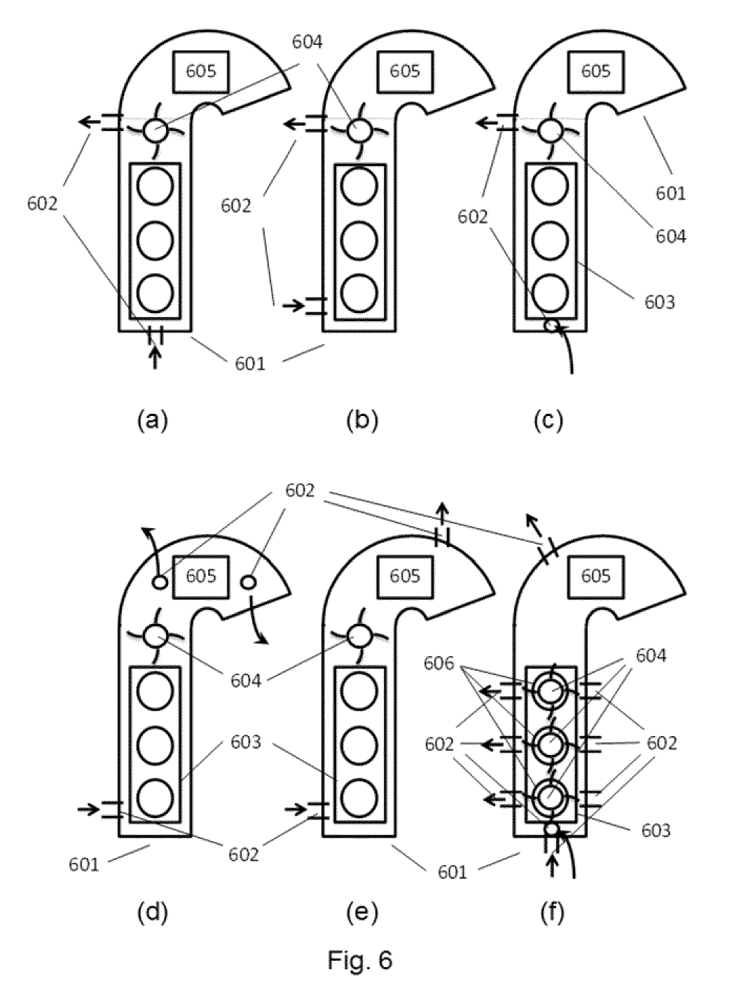

The position of the air holes 602 on the speech processor housing 601 relative to the battery pack 603, micro-ventilation mechanism 604, and/or processor 605 may also vary, in accordance with various embodiments of the invention, as shown in FIGS. 6(a-f). The air holes 602 may be, without limitation, positioned on a surface of the speech processor housing 601 that is averted away from the skin of the user.

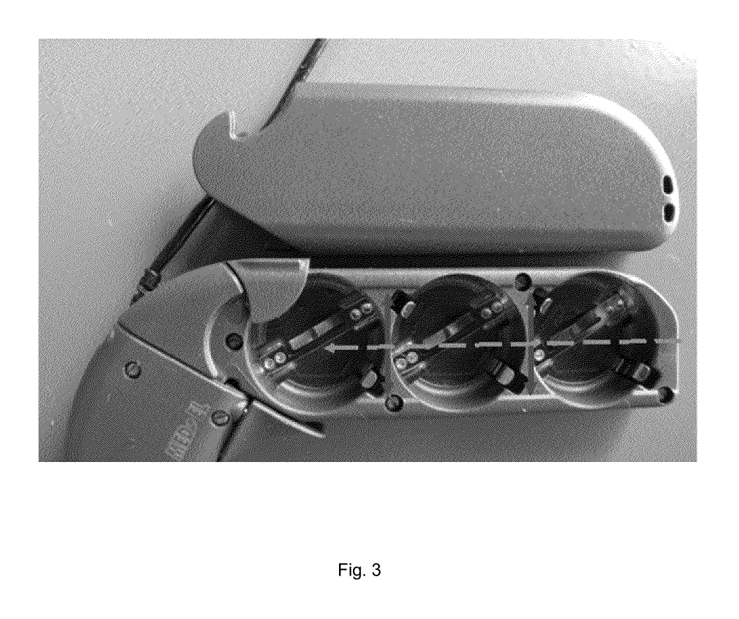

Illustratively, a speech processor of a cochlear prosthesis is shown similar to FIGS. 2 and 3 (with the battery pack 603 attached to the processor 605). More particularly, FIGS. 6(a-e) show the micro-ventilation mechanism 604 placed, without limitation, between the processor 605 and the battery pack 603. Additionally, and without limitation, FIG. 6(a) shows the air holes 602 positioned at the bottom of the battery pack 603 and on the side of the housing 601 adjacent to the micro-ventilation mechanism 604. FIG. 6(b) shows the air holes 602 positioned on the lower side of the battery pack 603 and on the side of the housing 601 adjacent to the micro-ventilation mechanism 604. FIG. 6(c) shows the air holes 602 positioned at the bottom corner of the battery pack 603 and on the side of the housing 601 adjacent to the micro-ventilation mechanism 604. FIG. 6(d) shows the air holes 602 positioned on the lower side of the battery pack 603 and on the front side of the housing 601 between the micro-ventilation mechanism 604 and the processor 605, and additionally, air holes 602 positioned on the front top of the speech processor housing 601 near the processor 605. FIG. 6(e) shows the air holes 602 positioned on the lower side of the battery pack 603 and on the top side of the housing 601 proximate the processor 605. FIG. 6(f) shows a plurality of micro-ventilation mechanisms 604, one for each battery 606 of the battery pack 603, with air holes 602 place proximate the bottom of the housing 601, proximate each micro-ventilation mechanism 604, and placed proximate the processor 605.

In accordance with further embodiments of the invention, power to the micro-ventilation mechanism may be provided by the battery(s), and/or by alternative energy sources. Alternative energy sources include, without limitation, solar cells which may be attached to the surface of the speech processor (or other external processor device), and/or a thermoelectric generator, which uses, for example, the temperature difference between body temperature and the environment.

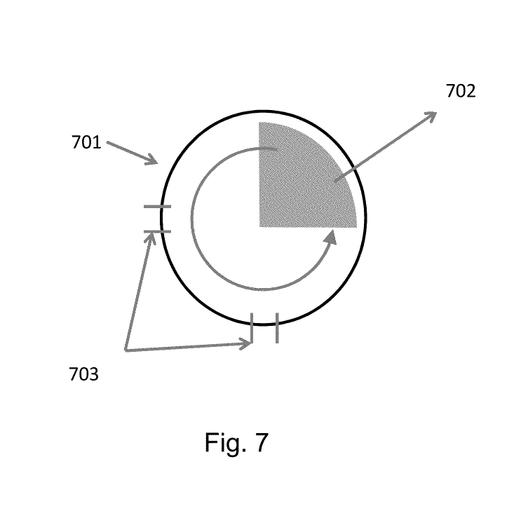

Alternatively, an electronically passive micro-ventilation mechanism 701 may be used to move air through the device. Various embodiments may include a rotatable mass 702, as shown in FIG. 7, or an arrangement of rotatable masses (e.g. a thin half or quarter of a cylinder, but many other geometries could be used) similar to that used in automatic watches. Illustratively, a rotatable mass 702 may wind a spring which in turn drives the ventilator, or a rotatable mass 702 itself soaks at each movement a sufficient volume of air to vent the batteries. The rotatable mass 702 may be installed in the medical device such that movement of the carrier's head (e.g. rocking the head) drives the rotatable mass. Part of the housing or battery holder may be free to move in a limited range. By this movement, it may suck in and/or pump out air of the inlet and/or outlet 703. For the use in a speech processor of a hearing aid, the rotatable mass 702 advantageously may have a mass below 1 g, preferable below 0.5 g and a size to fit within the device.

Advantages of a medical device that includes a micro-ventilation mechanism for moving air across batteries and/or various electronics include improved battery performance and efficiency due to improved air flow rate. Since there will always be sufficient air for the battery reaction, the efficiency increases. Additionally, since smaller and/or a less number of holes are necessary, there will be an increased freedom in the design of the battery pack Problems with dust, dirt or sweat can be minimized, and the lifetime of the device can be improved. In various embodiments, the micro-ventilation mechanism is very small so integration into the battery pack and/or a processor of the device is simplified, as variation in the dimensions of the battery pack may not necessary and weight will remain approximately the same. Advancements in Zn-air batteries, even rechargeable versions, are underway and may be incorporated in various embodiments of the invention.

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. A medical device comprising:
an external portion adapted for placement external to the skin of a user, the external portion including:
a battery pack for interfacing with at least one battery cell, the battery pack including:
a housing, the housing defining air inlet and/or outlet holes such that fluid flow is enabled through at least a part of the housing; and
a micro-ventilation mechanism for moving air through at least a part of the housing.

2. The medical device according to claim 1, further comprising an implantable portion that receives a signal from the external portion.

3. The medical device according to claim 2, wherein the external portion includes a first coil, and wherein the implantable portion includes a second coil, the first coil and the second coil for transcutaneous transmission of the signal via electromagnetic coupling.

4. The medical device according to claim 3, wherein the battery pack supplies a power signal to the first coil, for transcutaneous transmission to the second coil.

5. The medical device according to claim 2, wherein the implantable portion includes a stimulator module for producing for the auditory system of a user a stimulation representative of an acoustic signal.

6. The medical device according to claim 5, wherein the stimulation is an electrical stimulation and/or a mechanical stimulation.

7. The medical device according to claim 2, wherein the implantable portion includes one of a laryngeal pacemaker, a middle-ear implant and a cochlear implant.

8. The medical device according to claim 1, wherein the external portion further include a processor module, the micro-ventilation mechanism moving air across the processor module.

9. The medical device according to claim 1, wherein the micro-ventilation mechanism is a MEMS device.

10. The medical device according to claim 1, wherein the micro-ventilation mechanism acts as a fluid pump.

11. The medical device according to claim 1, wherein the micro-ventilation mechanism acts as a fan.

12. The medical device according to claim 1, wherein the micro-ventilation mechanism include a membrane.

13. The medical device according to claim 1, wherein the battery pack provides power to the micro-ventilation mechanism.

14. The medical device according to claim 1, wherein the external portion includes a solar cell for providing power to the micro-ventilation mechanism.

15. The medical device according to claim 1, wherein the external portion includes a thermoelectric generator module for providing power to the micro-ventilation mechanism.

16. The medical device according to claim 1, wherein the micro-ventilation mechanism is electronically passive.

17. The medical device according to claim 16, wherein the micro-ventilation system includes a movable mass.

18. The medical device according to claim 17, wherein the movable mass winds a spring.

19. The medical device according to claim 17, wherein the movable mass soaks at each movement a volume of air.

20. The medical device according to claim 17, wherein the movable mass rotates.

21. The medical device according to claim 17, wherein the movable mass is part of the housing.

22. The medical device according to claim 1, wherein the mass of the micro-ventilation mechanism is below 1 gram or below 0.5 gram.

23. The medical device according to claim 1, wherein the medical device is a hearing aid, a cochlear implant, and/or a laryngeal pacemaker.
